(19) United States
(12) Patent Application Publication
(10) Pub. No.: US 2010/0191151 A1
(43) Pub. Date: Jul. 29, 2010

(54) BIPOLAR ELECTRODE TYPE GUIDE WIRE AND CATHETER SYSTEM

(75) Inventor: Byung-Kuk Kwak, Seoul (KR)

Correspondence Address: LADAS & PARRY LLP, 224 SOUTH MICHIGAN AVENUE, SUITE 1600, CHICAGO, IL, 60604, US

(73) Assignees: TAEWOONG MEDICAL CO., LTD., Gyeonggi-do (KR); Chung-Ang University Industry-Academy Cooperation Foundation, Seoul (KR)

(21) Appl. No.: 12/664,692

(22) PCT Filed: Jun. 13, 2008
(86) PCT No.: PCT/KR2008/003354

§ 371 Date: Dec. 15, 2009

(30) Foreign Application Priority Data
Jun. 15, 2007 (KR) 10-2007-0058710
Apr. 15, 2008 (KR) 10-2008-0034645
Apr. 15, 2008 (KR) 10-2008-0034654

(51) Int. Cl. A61M 25/01

(52) U.S. Cl. 600/585

(57) ABSTRACT

Provided are a guide wire and a catheter system. The catheter system includes a hollow catheter inserted into a fistula of a living body, the catheter having two open ends and a coaxially-formed lumen; and a bipolar electrode type guide wire including a wire body formed of a metal and elongated to be inserted into the catheter in one direction, an insulating material coated on an outer periphery of the wire body to insulate the wire, a first electrode attached to one end of the wire body, a second electrode spaced a predetermined distance apart from the first electrode, a first lead elongated along the wire body to be attached to the first electrode to electrically connect a radio-frequency generator for generating radio-frequency current to the first electrode, and a second lead elongated along the wire body to be attached to the second electrode to electrically connect the radio-frequency generator to the second electrode, the second lead being electrically insulated from the first lead.

### TECHNICAL FIELD

The present invention relates to a guide wire and a catheter system, and more particularly, to a bipolar electrode type guide wire and a catheter system used for radio-frequency ablation such as vascular occlusion, removal of a tumor, tubular occlusion, fistula occlusion, shunt occlusion, and so on.

### BACKGROUND ART

In various cases, for example, vascular malformation such as arteriovenous malformation, hemorrhage disease due to rupture of organs, or the like, occlusion or embolization is needed to occlude a blood vessel of a bleeding portion.

In general, the conventional embolization occludes a blood vessel using an embolus material such as polyvinyl alcohol, gel foam, ethanol absolute, microsphere, coil, detachable balloon, and so on. After inserting a catheter into the affected part along the blood vessel, the embolus material is injected into the affected part through the catheter to occlude the blood vessel.

However, when the blood vessel is occluded using the embolus material, side effects due to the embolus material may be generated. That is, the embolus material may flow backward to block a normal blood vessel adjacent thereto, and the embolus material may be introduced into a vein to block a pulmonary artery, thereby causing iatrogenic embolism.

Meanwhile, a method of occluding a blood vessel through radio-frequency ablation (RFA) using radio-frequency current without using an embolus material is widely used. FIG. 1 is a schematic view showing radio-frequency ablation using a catheter, and FIGS. 2 to 4 are schematic view for explaining radio-frequency ablation for treating patients suffering from a bleeding disorder due to rupture of the kidney caused by injury using a catheter and an electrode, wherein FIG. 2 shows a state before treatment, FIG. 3 shows a state in which the catheter and a guide wire are inserted, and FIG. 4 shows a state after treatment.

In order to perform radio-frequency ablation, first, a catheter 1 should be inserted into a patient's body, and a proximal segment of the catheter should get close to an unwell area through a blood vessel. Since inserting the catheter 1 into the blood vessel is a very delicate operation, it is performed through an angiography system. When the proximal segment of the catheter arrives at the unwell area, power is applied to a radio-frequency generator (not shown) to apply radio-frequency current to the unwell area and thereby perform treatment, which will be described in detail. The catheter 1 has a hollow shape, in which a lumen (not shown) is formed. A guide wire (not shown) is coaxially inserted into the lumen of the catheter 1, and a distal end of the guide wire projects from the catheter 1 and is exposed. Therefore, when the proximal segment of the catheter 1 arrives at the unwell area, a proximal segment of the guide wire is also disposed at the same region as the catheter. The guide wire, formed of a metal, is electrically connected to the radio-frequency generator. Meanwhile, a ground pad (not shown) is attached to the patient's body (the patient's skin) and electrically connected to the radio-frequency generator. When power is applied to the radio-frequency generator, a current transmission path from the guide wire to the ground pad is formed. During transmission, friction due to ion oscillation increases the temperature of tissues and induces coagulation necrosis to thereby occlude the blood vessel.

FIG. 2 shows an example in which rupture of the kidney and pseudo-aneurysm of the renal artery were generated by an external injury. It will be appreciated that a large amount of hemorrhage occurred in a region indicated by reference character 2 of FIG. 2 due to the external injury. FIG. 3 shows a state in which a guide wire 3 inserted into the catheter 1 arrived at the hemorrhage region 2 through the blood vessel, and FIG. 4 shows the result of radio-frequency ablation using radio-frequency current performed at the hemorrhage region 2. Referring to FIG. 4, it will be appreciated that the hemorrhage was stopped and the blood vessel was effectively occluded through radio-frequency ablation.

As described above, radio-frequency ablation using the catheter system can prevent side effects such as occlusion of a normal blood vessel, which may be generated due to use of an embolus material. However, there may be side effects in normal organs such as the heart, nervous system, skin, and so on, in the current transmission path (from the unwell area to the ground pad), outside of the unwell area. In addition, the catheter system described above is inconvenient in that the ground pad must be attached to the patient to perform the treatment.

### DISCLOSURE OF INVENTION

### Technical Problem

In order to solve the foregoing and/or other problems, it is an object of the present invention to provide an improved bipolar electrode type guide wire and catheter system which can very effectively perform radio-frequency ablation by locally inducing coagulation necrosis only in a region affected by a medical condition.

### Technical Solution

One aspect of the present invention provides a bipolar electrode type guide wire including: a first wire formed of an elongated electrically conductive material, and inserted into a hollow catheter, which is inserted into a fistula of a living body, to project from the catheter at both ends thereof; and a second wire formed of an elongated electrically conductive material, and including a main wire part spaced apart from the first wire, and a coil part extending from the main wire part in a spiral shape, the first wire being inserted into the coil part, wherein the first wire is electrically insulated from the second wire, front ends of the first and second wires are not insulated, and the non-insulated front end of the first wire is spaced a predetermined distance apart from the non-insulated front end of the second wire.

### ADVANTAGEOUS EFFECTS

In accordance with the present invention, it is possible to perform radio-frequency ablation by locally inducing coagulation necrosis only in a region affected by a medical condition such as malformation of a blood vessel, a tumor, hemorrhage, and so on, without side effects such as rupture of normal tissues, and so on, has a simple and economical constitution, and is convenient to operate.

### BRIEF DESCRIPTION OF THE DRAWINGS

The above and other objects, features and advantages of the present invention will become apparent by describing certain exemplary embodiments thereof with reference to the attached drawings, in which:

FIG. 1 is a schematic view showing radio-frequency ablation using a catheter;

FIGS. 2 to 4 show photographs for explaining radio-frequency ablation for treating patients suffering from a bleeding disorder due to rupture of the kidney caused by injury using a catheter and an electrode, wherein FIG. 2 shows a state before treatment,

FIG. 3 shows a state in which the catheter and a guide wire are inserted, and FIG. 4 shows a state after treatment;

FIG. 5 is a schematic perspective view of a bipolar electrode type guide wire and a catheter system in accordance with a first exemplary embodiment of the present invention;

FIG. 6 is a schematic cross-sectional view taken along line VI-VI of FIG. 5;

FIG. 7 is a schematic cross-sectional view taken along line VII-VII of FIG. 6;

FIG. 8 is a schematic perspective view of a bipolar electrode type guide wire in accordance with a second exemplary embodiment of the present invention;

FIG. 9 is a schematic perspective view of a bipolar electrode type guide wire in accordance with a third exemplary embodiment of the present invention;

FIG. 10 is a schematic perspective view of a bipolar electrode type guide wire and a catheter system in accordance with a fourth exemplary embodiment of the present invention;

FIG. 11 is a schematic cross-sectional view taken along line XI-XI of FIG. 10;

FIG. 12 is a schematic cross-sectional view taken along line XII-XII of FIG. 11;

FIG. 13 is a schematic cross-sectional view taken along line XIII-XIII of FIG. 11;

FIG. 14 is a perspective view of a bipolar electrode type guide wire in accordance with a fifth exemplary embodiment of the present invention;

FIG. 15 is a perspective view of a bipolar electrode type guide wire in accordance with a sixth exemplary embodiment of the present invention;

FIG. 16 is a partially cut perspective view of a catheter system in accordance with a seventh exemplary embodiment of the present invention;

FIG. 17 is a schematic cross-sectional view taken along line XVII-XVII of FIG. 16;

FIG. 18 is a schematic cross-sectional view taken along line XVIII-XVIII of FIG. 17;

FIG. 19 is a perspective view for explaining a shape holding body for an electrode of a catheter system in accordance with an eighth exemplary embodiment of the present invention;

FIG. 20 is a perspective view for explaining a shape holding body for an electrode of a catheter system in accordance with a ninth exemplary embodiment of the present invention;

FIG. 21 is an exploded perspective view for explaining an electrode member in accordance with a tenth exemplary embodiment of the present invention; and

FIG. 22 is a cross-sectional view taken along line XXII-XXII of FIG. 21.

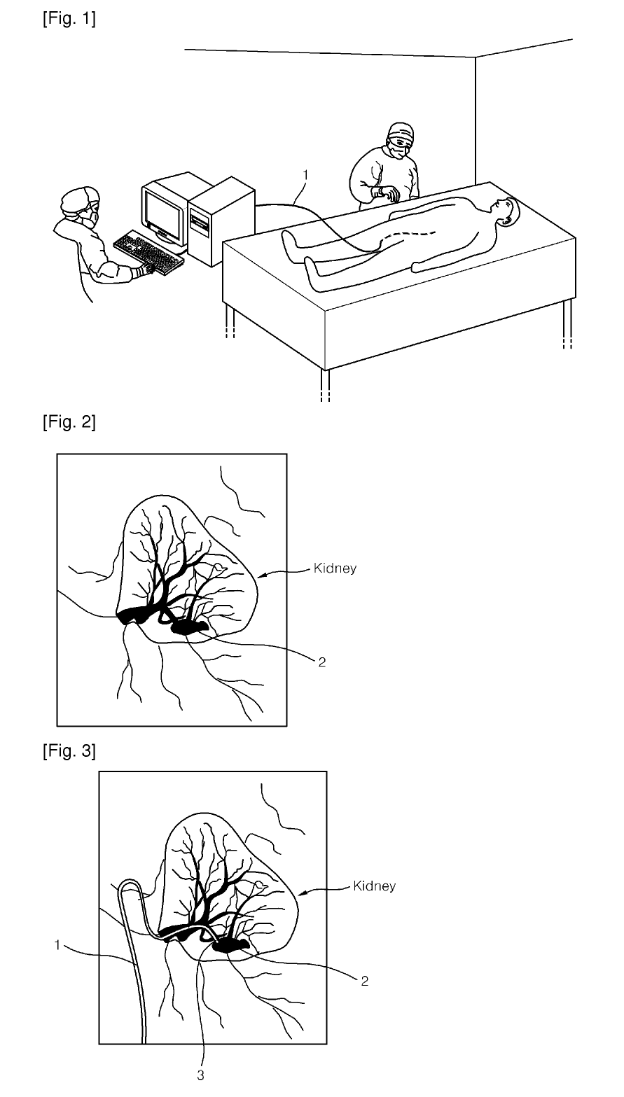

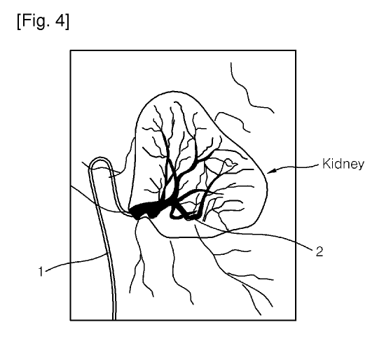

### MODE FOR THE INVENTION

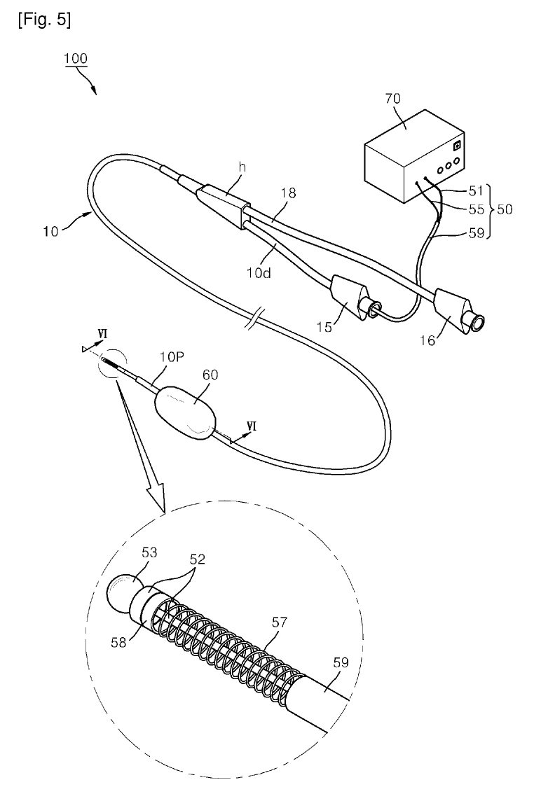

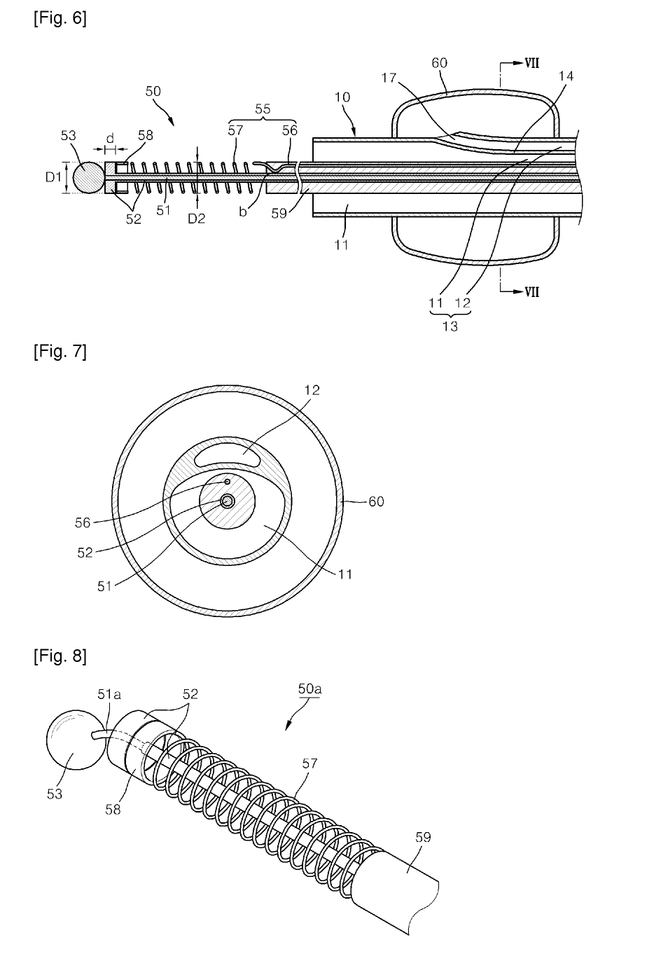

FIG. 5 is a schematic perspective view of a bipolar electrode type guide wire and a catheter system in accordance with a first exemplary embodiment of the present invention, FIG. 6 is a schematic cross-sectional view taken along line VI-VI of FIG. 5, FIG. 7 is a schematic cross-sectional view taken along line VII-VII of FIG. 6.

Referring to FIGS. 5 to 7, the catheter system 100 in accordance with an exemplary embodiment of the present invention includes a catheter 10, a bipolar electrode type guide wire 50, a balloon 60, and a radio-frequency generator 70.

The catheter 10 has an elongated shape and a substantially circular cross-section, and is inserted into a fistula of a living body, for example, a blood vessel. The catheter 10 is generally classified according to its thickness as either a general catheter or a micro catheter which is very thin (0.3 mm to 0.5 mm). The length of the catheter 10 varies but is formed in the range of 80 cm to 160 cm. Since the catheter 10 is inserted into a human body through a blood vessel, it is formed of a flexible material. The flexible material may include polytetrafluoroethylene (PTFE), known as Teflon resin, or fluorine resin such as hexafluoropropylene copolymer (FEP), perfluoroalkylvinylether copolymer (PFA), and so on. PTFE, a crystalline polymer having a melting point of 327° C., has a continuous use temperature of 260° C. and can be stably used from a low temperature of −268° C. to a high temperature. In addition, PTFE has strong chemical resistance so as not to react with various solvents such as acidic solvent, basic solvent, etc., and thereby be used stably. Further, the flexible material may include polyethylene, polystyrene, polyurethane, and so on. Such fluorine resin functions to reduce friction upon insertion of the bipolar electrode type guide wire 50, such that the guide wire 50 can be readily inserted into the catheter 10. Meanwhile, an outer periphery of the catheter 10 is coated with a hydrophilic material such that the catheter can be readily inserted into a blood vessel.

The catheter 10 has a hollow shape, two open ends, and a lumen 13. The lumen 13 is coaxially disposed in a longitudinal direction of the catheter 10. In addition, in this exemplary embodiment, the lumen 13 is divided into a first lumen 11 and a second lumen 12 by a diaphragm 14 longitudinally formed along the catheter 10. The first lumen 11 includes both ends of the catheter 10, i.e., a proximal end 10p inserted into a human body, and a distal end 10d disposed outside the human body, to pass through the entire catheter 10. The first lumen 11 functions as a passage for inserting the bipolar electrode type guide wire 50, which will be described. The second lumen 12 functions as a passage for injecting fluid into the balloon 60, which will be described, and extends from a portion of the catheter 10 in which the balloon 60 is disposed to the distal end 10d opposite to the proximal end 10p of the catheter 10. Therefore, an inlet port 17 is formed at one end of the second lumen 12 to pass through between the inner periphery and the outer periphery of the catheter 10 such that the fluid is introduced into the balloon 60. In addition, a fluid injection tube 18 is disposed at the other end of the second lumen 12. The fluid injection tube 18 also has a hollow shape in which a third lumen (not shown) is coaxially formed. The third lumen guides the fluid injected into the balloon 60 toward the second lumen 12, and the third lumen is in communication with the second lumen 12. The fluid injection tube 18 is inserted into and coupled to a hub h, into which the catheter 10 is fixedly inserted. The second lumen 12 and the third lumen are connected to each other in the hub h.

In addition, an insertion hub 15 having an insertion hole is installed at the distal end 10d of the catheter 10 such that the bipolar electrode type guide wire 50 can be readily inserted. The insertion hole is tapered such that the diameter decreases toward the proximal end 10p. Similarly, an injection hub 16 in which an injection hole is formed at an angle is installed at an end of the fluid injection tube 18 to readily inject the fluid.

The bipolar electrode type guide wire 50 includes a first wire 51 and a second wire 55.

The first wire 51, which is long, is coaxially inserted into the first lumen 11 of the catheter 10. In addition, both ends of the first wire 51 are disposed to project from the catheter 10. In the bipolar electrode type guide wire 50 and the catheter system 100 in accordance with an exemplary embodiment of the present invention, the first wire 51 performs a basic function as a path guide to the unwell area along the blood vessel, and a function as an electrode for radio-frequency ablation. In order to perform its function as an electrode, the first wire 51 is formed of a metal having electrical conductivity.

A first electrode 53 having a spherical shape is attached to a front end of the first wire 51. The first spherical electrode 53 formed of an electrically conductive material is disposed at the front end of the first wire 51 to be electrically operated with the second electrode 58 attached to a front end of the second wire 55.

In addition, an outer periphery of the first wire 51 is coated with a polymer material as an insulating material, for example, a coating material 52 like Teflon resin. More specifically, except the front end of the region of the first wire 51 projecting from the first lumen 11 of the catheter 10 and a rear end connected to the radio-frequency generator 70, the entire center part of the first wire 51 is coated with the coating material 52. As described above, since the front end of the first wire 51, i.e., the first electrode 53, acts as an electrode for conducting current to the front end of the second wire 55, and the rear part of the first wire 51 is electrically connected to the radio-frequency generator 70, the front and rear ends of the first wire 51 should not be insulated by the coating material 52.

Meanwhile, since the part of the first wire 51 projecting from the lumen of the catheter 10 should be inserted into microvasculature, that part is formed of a material such as platinum, and so on, to have greater flexibility than the other parts.

The second wire 55 is also coaxially inserted into the lumen of the catheter 10, like the first wire, and includes a main wire part 56 and a coil part 57. Since the second wire 55 also acts as an electrode, like the first wire 51, the second wire 55 is formed of a metal having electrical conductivity.

The main wire part 56 is long and runs parallel to, spaced a predetermined distance apart from, the first wire 51. The coil part 57 extends from an end of the main wire part 56 in a spiral shape. A second electrode 58 having a ring shape is attached to an end of the coil part 57, i.e., the front end of the second wire 55. The second electrode 58 is formed of an electrically conductive material. Since the ring-shaped second electrode 58 acts as an electrode corresponding to the first electrode 53 of the first wire 51, a path of alternating current between the first and second electrodes 53 and 58 is formed.

In addition, the first wire 51 is inserted into the coil part 57. However, since the first electrode 53 of the first wire 51 is disposed to project from the coil part 57, the first electrode 53 (the front end of the first wire 51) is spaced a predetermined distance d from the second electrode 58 (the front end of the second wire 55). The distance d may be 1 mm to 50 mm. While described below, the unwell area such as a blood vessel or a tumor is located between the front end of the first wire 51 and the front end of the second wire 55, and alternating current flows between the front ends of the first and second wires 51 and 55 to occlude the blood vessel or cauterize the tumor. When the distance between the front ends of the first and second wires 51 and 55, i.e., the distance d between the first and second electrodes 53 and 58, is 1 mm or less, the resulting current propagation region is very small, which makes it difficult to effectively treat the unwell area, and the front ends may easily come into contact. And when the distance d is more than 50 mm, current cannot flow smoothly, which makes it difficult to perform the effective treatment, and normal tissues in the vicinity of the unwell area may be adversely affected.

Since the bipolar electrode type guide wire 50 in accordance with an exemplary embodiment of the present invention is inserted into the blood vessel as described above, it is not preferable for the guide wire 50 to have a step. Therefore, the diameter D1 of the first wire, the outer diameter of the second electrode 58, and the outer diameter D2 of the coil part 57 may be the same. In addition, in order to prevent formation of a step in the guide wire 50 and maintain a uniform diameter in accordance with an exemplary embodiment of the present invention, the coating material disposed between the first electrode 53 and the second electrode 58 may be thicker than other parts. That is, the coating material may have the same outer diameter as the first electrode 53 and the second electrode 58.

While not employed in the present exemplary embodiment, like the first wire 51, an outer periphery of the main wire part 56 of the second wire 55 may be coated with a coating material such as Teflon. However, in order to prevent an increase in thickness of the bipolar electrode type guide wire 50 in accordance with an exemplary embodiment of the present invention, the coil part 57 of the second wire 55 is not coated. In addition, since the front ends of the first and second wires 51 and 55 act as electrodes, and the rear end of the second wire 55 is electrically connected to the radio-frequency generator 70, none of these ends should be insulated. Therefore, the front ends of both wires and the rear end of the second wire are not coated with the coating material.

As described above, while the first wire 51 and the second wire 55 may be electrically connected to each other by coating the first wire 51 and the main wire part of the second wire 55 using Teflon and so on, in an exemplary embodiment of the present invention, the first wire 51 and the main wire part 56 of the second wire 55 are electrically insulated from each other by a covering material 59 such as a polymer material. That is, in a state in which the first wire 51 is spaced apart from the main wire part 56 of the second wire 55, the covering material 59 formed of a melted polymer material surrounds the first wire 51, the main wire part 56 of the second wire 55, and therebetween, and after a predetermined time elapses, the liquid covering material 59 solidifies.

In this state, the covering material 59 formed of a polymer is interposed between the first and second wires 51 and 55 to electrically connect the wires. In addition, since the first and second wires 51 and 55 are physically connected by the covering material 59, they can be more readily used or operated than if they were separated from each other. However, since it is not preferable for the first wire 51 and the second wire 55 to move relative to one another when the guide wire 50 in accordance with the present invention is inserted into the blood vessel, the first wire 51 and the main wire part 56 of the second wire 55 are combined with each other. Since the outer periphery of the first wire 51 is surrounded by the coating material 52 and is electrically insulated from the main wire part 56 of the second wire 55, relative movement between the first wire 51 and the main wire part 56 of the second wire 55 coated with the coating material 52 is prevented by an adhesive b or a coupling member (not shown).

Meanwhile, since the outer diameter of the covering material 59 is equal to the diameter D2 of the coil part 57 of the second wire 55, the coil part 57 and the covering material 59 have the same diameter, without any step.

While the bipolar electrode type guide wire 50 may have various diameters depending on its use, in this exemplary embodiment, the coil part 57 of the second wire 55 is about 0.016 inches in diameter, and the catheter 10 is 0.038 inches or more in outer diameter.

The balloon 60 is hermetically sealed with the outer periphery of the catheter 10. More specifically, the balloon 60 surrounds a region of the inlet port 17 of the second lumen 12 and is coupled to the outer periphery of the catheter 10. The balloon 60 formed of a flexible material expands in the blood vessel to block blood flow. That is, fluid (generally, used for angiography) is introduced between the inner periphery of the balloon 60 and the outer periphery of the catheter 10 through the inlet port 17 via the second lumen 12 to expand the balloon 60. The proximal end 10p of the catheter 10 is in a shrunk state until it arrives at the unwell area and it expands when the fluid is injected. The balloon 60 may be formed of an antithrombogenic material having good thermal resistance, because its outer surface is in contact with blood and a large amount of heat is generated during radio-frequency ablation.

The radio-frequency generator 70 generates radio-frequency alternating current to be used in electro-surgery for locally cutting or coagulating living tissues. In this exemplary embodiment, the first wire 51 of the bipolar electrode type guide wire 50 is electrically connected to a positive terminal (+) of the radio-frequency generator 70, and the second wire 55 is electrically connected to a negative terminal (−) of the radio-frequency generator 70. Therefore, this exemplary embodiment employs a bipolar electrode in which the first wire 51 and the second wire 55 act as a positive electrode and a negative electrode, not a monopolar electrode using a ground pad as in the conventional art. When about 20 Watts of power is applied to the radio-frequency generator 70, alternating current in a radio-frequency region (200 to 1200 kHz) flows between the first electrode 53 of the first wire 51 and the second electrode 58 of the second wire 55. In the process of generating alternating current, friction due to ion oscillations increases a temperature of living tissues such as blood vessels, tumors, etc., to induce coagulation necrosis and thereby occlude the blood vessel or cauterize the tumor.

When radio-frequency ablation using the monopoler electrode is performed, as described above, attachment of the ground pad to a patient causes inconvenience and elongates the current transmission path from the electrode to the ground pad, adversely affecting other important blood vessels or tissues in the vicinity of the blood vessel to be occluded. However, when the bipolar electrode in accordance with the present invention is used, the current transmission path is locally formed at the bipolar electrode type guide wire 50 only to prevent side effects in other tissues and blood vessels.

In addition, the balloon 60 enables effective radio-frequency ablation even when blood flow through the bleeding part is fast. That is, ablation through the radio-frequency generator 70 induces coagulation by increasing the temperature of the tissues. When blood flow is fast, a phenomenon in which heat is carried away with the blood flow rather than being radiated into the unwell area (conventionally, referred to as a “heat sink effect” is generated and makes it difficult to induce coagulation necrosis. In this case, effective treatment can be performed by expanding the balloon 60 in the blood vessel to temporarily block the blood flow and perform radio-frequency ablation.

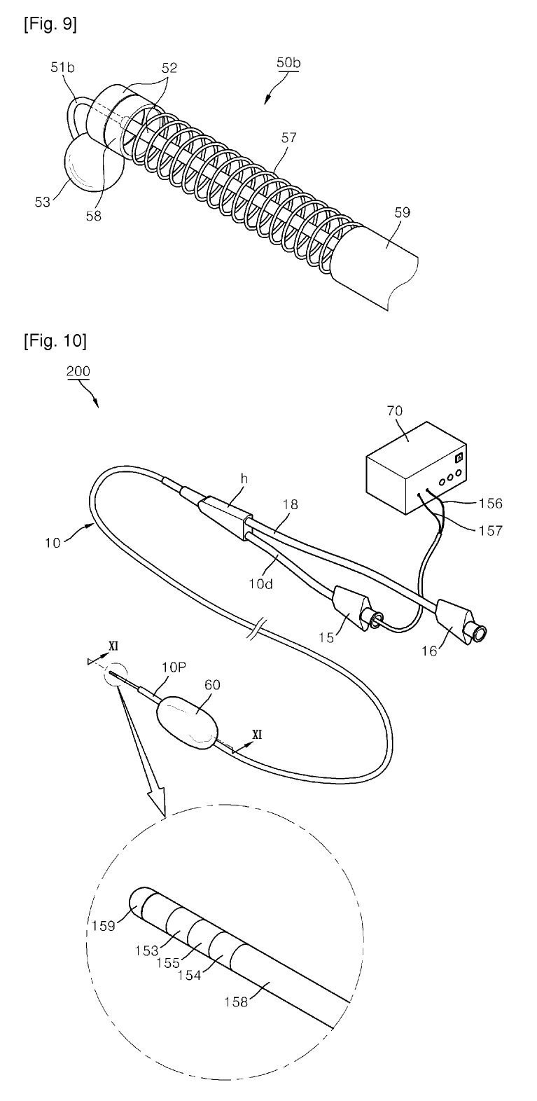

While the first exemplary embodiment has been described as employing the bipolar electrode type guide wire 50 including the first wire 51 having one straight end (adjacent to the proximal end 10p of the catheter), the one end of the first wire may have various shapes. FIGS. 8 and 9 show an exemplary embodiment in which the first wire 51 has one curved end.

FIG. 8 is a schematic perspective view of a bipolar electrode type guide wire in accordance with a second exemplary embodiment of the present invention, and FIG. 9 is a schematic perspective view of a bipolar electrode type guide wire in accordance with a third exemplary embodiment of the present invention.

Referring to FIG. 8, one end of a first wire 51a is bent across the longitudinal direction of the catheter. In addition, referring to FIG. 9, one end of a first wire 51b is entirely bent to form a “U” shape. A wide blood vessel branches into narrow blood vessels such that the branching blood vessels are disposed in directions crossing the wide blood vessel. When an operator intends to insert the bipolar electrode type guide wire 50a into a branching blood vessel while moving along the wide blood vessel, it is advantageous that one end of the first wire 51a is bent as shown in FIG. 8. That is, when the bipolar electrode type guide wire 50a is rotated at the branching blood vessel, the bent end of the first wire 51a can be directed into the branching blood vessel.

In addition, when the blood vessel branches in a reverse direction and the bipolar electrode type guide wire 50b is inserted into the reversely branching blood vessel, as shown in FIG. 9, the first wire 51b having the U-shaped end can be readily inserted into the reversely branching blood vessel.

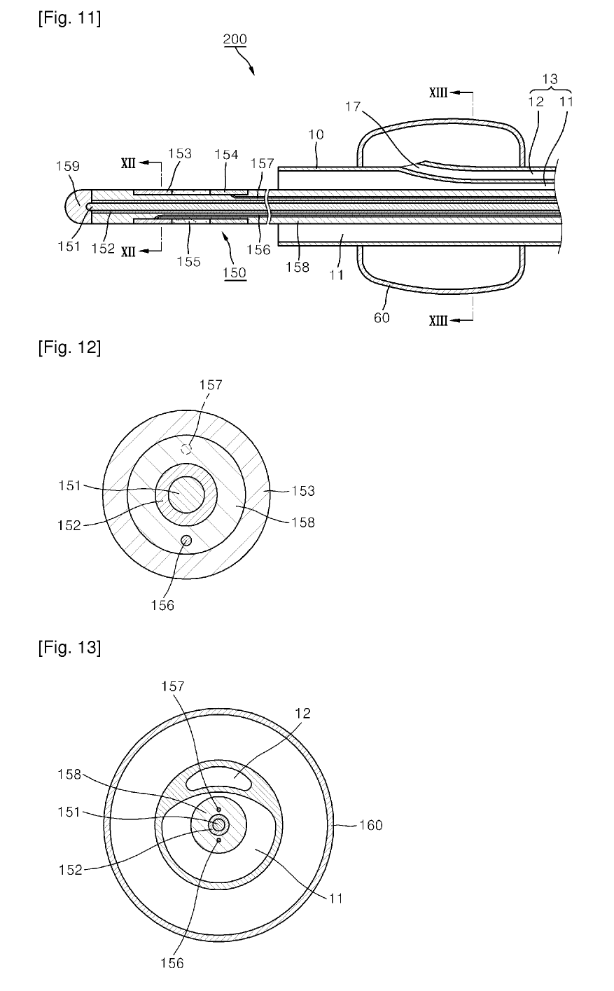

Meanwhile, the bipolar electrode type guide wire may have the following structure. FIG. 10 is a schematic perspective view of a bipolar electrode type guide wire and a catheter system in accordance with a fourth exemplary embodiment of the present invention, FIG. 11 is a schematic cross-sectional view taken along line XI-XI of FIG. 10, FIG. 12 is a schematic cross-sectional view taken along line XII-XII of FIG. 11, FIG. 13 is a schematic cross-sectional view taken along line XIII-XIII of FIG. 11.

Referring to FIGS. 10 to 13, the bipolar electrode type guide wire 150 in accordance with an exemplary embodiment of the present invention includes a wire body 151, a first electrode 153, and a second electrode 154.

The wire body 151 is formed of a flexible material and is long to be inserted into the blood vessel in a state in which it is inserted into a first lumen 11. In addition, the wire body 151 is formed of a metal and its position in the blood vessel can be recognized by an operator through an angiography system in order to help navigate the blood vessel to the unwell area. In addition, an insulating cap 159 formed of an insulating material is coupled to the wire body 151 to insulate a distal end thereof.

While the wire body 151 is shown to have a straight shape in the drawings, a front end of the wire body 151 actually has a coil shape enabling it to flex for easy movement through a narrow blood vessel. In addition, the front end of the wire body 151 is formed of a flexible material such as platinum, unlike other parts. The outer periphery of the wire body 151 is coated with a natural polymer material, for example, an insulating material 152 formed of Teflon resin, to insulate the wire body 151.

The first electrode 153 has a ring shape and is coupled with an outer periphery of one end of the wire body 151. However, the insulating material 152 and a covering material 158, which is to be described, are interposed between the first electrode 153 and the wire body 151, to electrically insulate the first electrode 153 from the wire body 151.

The second electrode 154 has a ring shape, like the first electrode 153, is spaced a predetermined distance apart from the first electrode 153, and is coupled with the outer periphery of the one end of the wire body. Similarly, the insulating material 152 and the covering material 158 are interposed between the second electrode 154 and the wire body 151 to electrically insulate the second wire 153 from the wire body 151.

The first electrode 153 is electrically connected to the radio-frequency generator 70 by a first lead 156. That is, the first lead 156 is elongated along the wire body 151 such that its one end is connected to the first electrode 153 and the other end is connected to a positive terminal (+) of the radio-frequency generator 70.

In addition, the second electrode 154 is electrically connected to the radio-frequency generator 70 by a second lead 157. Similar to the first lead 156, the second lead 157 is elongated along the wire body 151 such that its one end is connected to the second electrode 154 and the other end is connected to a negative terminal (−) of the radio-frequency generator 70.

The first lead 156 and the second lead 157 are insulated from each other by the covering material 158. That is, in a state in which the first lead 156 is spaced apart from the second lead 157, the covering material 158 formed of a melted polymer material surrounds the entire wire body 151 including the first lead 156 and the second lead 157, and is solidified when a predetermined time elapses. In this state, the polymer covering material 158 separates the first lead 156 and the second lead 157 from each other to electrically insulate the first and second leads 156 and 157.

Further, in order to prevent formation of a step at the outer periphery of the guide wire 150 by the first electrode 153 and the second electrode 154, the outer diameters of the first electrode 153 and the second electrode 154 are equal to the outer diameter of the covering material 158. In addition, a ring-shaped insulator 155 having the same outer diameter is inserted between the first electrode 153 and the second electrode 154, spaced apart from each other. Therefore, the bipolar electrode type guide wire 150 in accordance with an exemplary embodiment of the present invention has an entirely smooth outer periphery without any step.

Further, as described above, the unwell area such as a blood vessel, a tumor, or the like, is disposed between the first electrode 153 and the second electrode 154. In order to perform effective treatment, a spacing distance between the first electrode 153 and the second electrode 154 is 1 mm to 50 mm.

When the guide wire 150 in accordance with an exemplary embodiment of the present invention is used, like in the above exemplary embodiment, it is possible to prevent inconvenience in use such as attachment of the ground pad to a patient. In addition, since the transmission path is locally formed only between the first electrode 153 and the second electrode 154, it is possible to prevent side effects in other tissues or blood vessels.

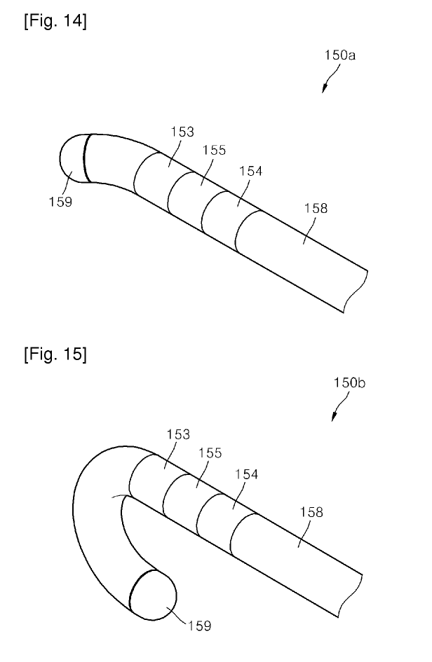

Meanwhile, unlike the fourth exemplary embodiment, the front end of the guide wire 150 may have a curved shape like the second and third exemplary embodiments. FIG. 14 is a perspective view of a bipolar electrode type guide wire in accordance with a fifth exemplary embodiment of the present invention, and FIG. 15 is a perspective view of a bipolar electrode type guide wire in accordance with a sixth exemplary embodiment of the present invention.

Referring to FIG. 14, the front end of the guide wire 150a in accordance with a fourth exemplary embodiment of the present invention is bent in a direction crossing the longitudinal direction of the catheter. In addition, referring to FIG. 15, the front end of the guide wire 150b is entirely bent to form a “U” shape. As described above, when the front end of the guide wire has a curved shape, as described in the second and third exemplary embodiments, the guide wire can be readily inserted into branches of the blood vessel.

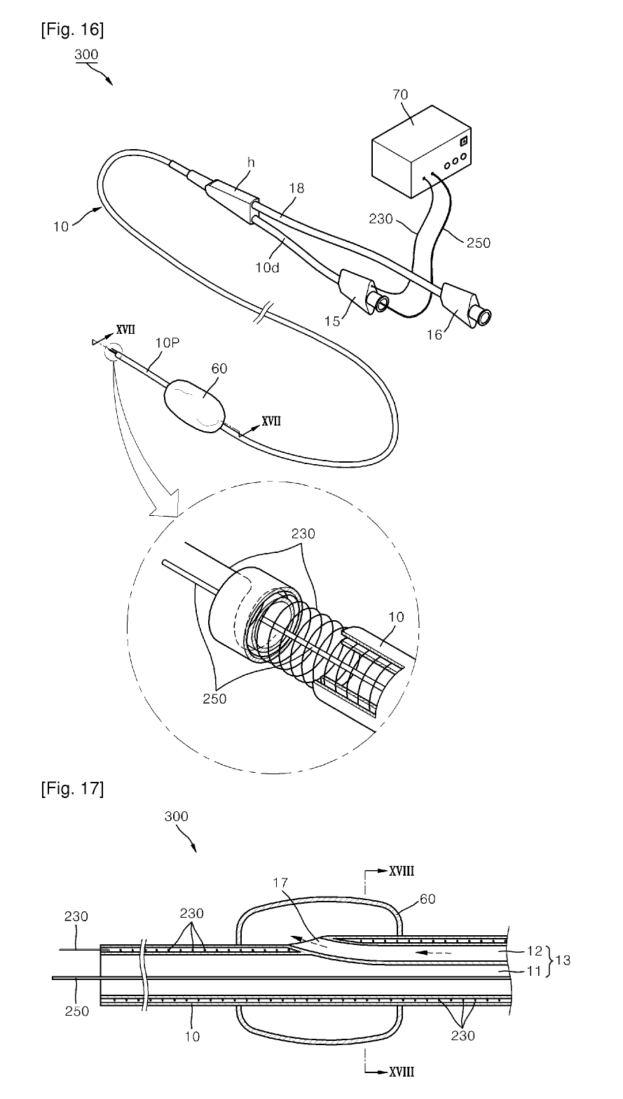

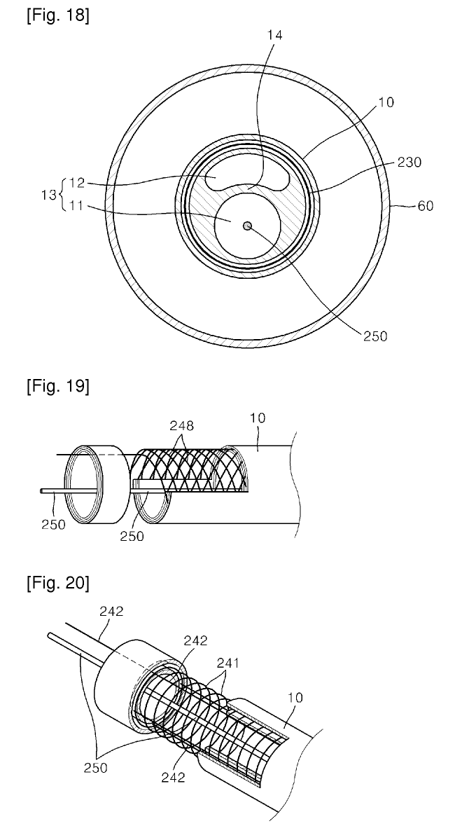

Meanwhile, unlike the above exemplary embodiments, the catheter system may be configured to have a shape holding body for an electrode. FIG. 16 is a partially cut perspective view of a catheter system in accordance with a seventh exemplary embodiment of the present invention, FIG. 17 is a schematic cross-sectional view taken along line XVII-XVII of FIG. 16, FIG. 18 is a schematic cross-sectional view taken along line XVIII-XVIII of FIG. 17.

Referring to FIGS. 16 to 18, the catheter system 300 in accordance with an exemplary embodiment of the present invention includes a catheter 10, a shape holding body 230 for an electrode, a guide wire 250, a balloon 60, and a radio-frequency generator 70. Here, since the catheter 10, the balloon 60 and the radio-frequency generator 70 are the same as in the above-described exemplary embodiments, they will not be described again. Rather, the shape holding body 230 for an electrode and the guide wire 250 will be described in detail.

The shape holding body 230 for an electrode functions to hold a shape of a lumen 13 formed inside the catheter 10. That is, since the catheter 10 is formed of a flexible material, the shapes of the first lumen 11 and the second lumen 12 may be deformed by a small external force. When the diameter of the first lumen 11 becomes smaller at a portion thereof due to an external force, the guide wire 250 cannot be readily inserted, and when the diameter of the second lumen 12 becomes smaller, it is difficult to inject fluid into the balloon 60. Thus, the shape holding body 230 is inserted between the inner periphery and the outer periphery of the catheter 10 and disposed along the entire length of the catheter 10 to prevent deformation of the first and second lumens 11 and 12 and hold their original shapes. In this exemplary embodiment, the shape holding body 230 for an electrode is wound between the inner periphery and the outer periphery of the catheter 10 in a spiral shape.

In addition, in this exemplary embodiment, the shape holding body 230 for an electrode functions to hold the shape of the lumen and acts as an electrode. Therefore, the shape holding body 230 for an electrode is formed of an electrically conductive material such as copper, stainless steel, and so on. Both ends of the shape holding body 230 for an electrode project from the catheter 10 to be exposed to the exterior, and one end thereof is electrically connected to a negative terminal of the radio-frequency generator 70.

The guide wire 250 is coaxially inserted into the first lumen 11 of the catheter 10 to guide a path through which the catheter 10 arrives at the unwell area along the blood vessel. The guide wire 250 is formed of a metal having electrical conductivity. In addition, the outer periphery of the guide wire 250 is coated with a polymer material, for example, an insulating material formed of Teflon resin. More specifically, a center part between one end of the guide wire 250, i.e., a portion in which the guide wire 250 projects from the lumen of the catheter 10, and the other end connected to the radio-frequency generator, is coated. The length of the guide wire 250 projecting from the lumen is about 0.5 to 2 cm. When the guide wire 250 is inserted into the first lumen 11 of the catheter 10, as described above, one end of the guide wire 250 is disposed to project beyond the first lumen 11. Therefore, one end of the guide wire 250 and one end of the shape holding body 230 for an electrode project from the catheter 10 and are spaced apart from each other. The other end of the guide wire 250 is electrically connected to both end terminals of the radio-frequency generator 70.

When the catheter system 300 in accordance with an exemplary embodiment of the present invention is used, as described in the aforementioned exemplary embodiment, it is possible to prevent inconvenience in use such as attachment of the ground pad to a patient. In addition, since the transmission path is locally formed between the end of the guide wire 250 and the end of the shape holding body 230 for an electrode, it is possible to prevent side effects in other tissues and blood vessels.

Meanwhile, the shape holding body for an electrode may have different shapes. FIG. 19 is a perspective view for explaining a shape holding body for an electrode of a catheter system in accordance with an eighth exemplary embodiment of the present invention, FIG. 20 is a perspective view for explaining a shape holding body for an electrode of a catheter system in accordance with a ninth exemplary embodiment of the present invention.

Referring to FIG. 19, the shape holding body 248 for an electrode of the eighth exemplary embodiment is disposed in a net shape and inserted between the inner periphery and the outer periphery of the catheter 10. In addition, a single cord of iron core of the net shape projects from the exterior of the catheter 10.

Referring to FIG. 20, the shape holding body for an electrode in accordance with a ninth exemplary embodiment of the present invention includes annular support bodies 241 and linear support bodies 242. The annular support bodies 241 are disposed in the longitudinal direction of the catheter 10 at predetermined intervals. In this exemplary embodiment, the linear support bodies 242 are four straight iron cores disposed in a circumferential direction of the annular support frames 241 at predetermined angular intervals (about 90° in the longitudinal direction of the catheter 10. One end of a single cord of the linear support bodies 242 projects from the catheter 10, and the other end is connected to the radio-frequency generator.

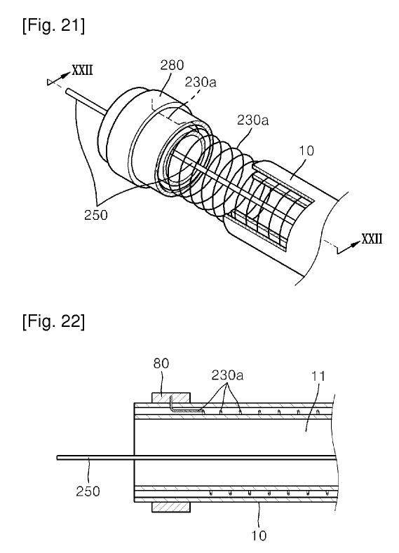

In addition, while it has been described that the shape holding body for an electrode itself acts as an electrode, a separate electrode member may be used. Such an exemplary embodiment is shown in FIGS. 21 and 22. FIG. 21 is an exploded perspective view for explaining an electrode member in accordance with a tenth exemplary embodiment of the present invention, and FIG. 22 is a cross-sectional view taken along line XXII-XXII of FIG. 21.

Referring to FIGS. 21 and 22, a ring-shaped electrode member 280 is coupled with one end of the catheter 10. The ring-shaped electrode member 280 is formed of an electrically conductive material. The shape holding body 230a spirally wound between the inner periphery and the outer periphery of the catheter 10 is electrically connected to the electrode member 280. Therefore, when power is applied, a current transmission path from the guide wire 250 to the ring-shaped electrode member 280 is formed. The ring-shaped electrode member 280 is spaced a predetermined distance apart from a tip of one end of the catheter 10 toward the other end of the catheter 10, or right at the tip of the one end of the catheter 10. However, when the spacing is too great, the current transmission path is also too long.

While the above exemplary embodiments have been described as using the radio-frequency generator, a microwave generator, etc. may be used according to the patient's condition.

In addition, while the above exemplary embodiments have been described as employing a structure in which the balloon 60 is coupled to the catheter 10, a catheter to which no balloon is attached may be used. In this case, there is no second lumen 12 or fluid injection pipe 18 for injecting fluid into the balloon.

As can be seen from the foregoing, since radio-frequency ablation can be concentrated on the unwell area, it is possible to perform very effective treatment without side effects in other tissues and blood vessels.

In addition, inconvenience due to attachment of a conventional ground pad can be eliminated.

Further, one end of a guide wire is bent so that it can be readily inserted into a branching blood vessel.

Although the present invention has been described with reference to certain exemplary embodiments thereof, it will be understood by those skilled in the art that various modifications can be made to the present invention without departing from the spirit or scope of the present invention defined in the appended claims and their equivalents.

1. A bipolar electrode type guide wire comprising:
a first wire formed of an elongated electrically conductive material, and inserted into a hollow catheter, which is inserted into a fistula of a living body, to project from the catheter at both ends thereof; and
a second wire formed of an elongated electrically conductive material, and
including a main wire part spaced apart from the first wire, and a coil part extending from the main wire part in a spiral shape, the first wire being inserted into the coil part,
wherein the first wire is electrically insulated from the second wire, front ends of the first and second wires are not insulated, and the non-insulated front end of the first wire is spaced a predetermined distance apart from the non-insulated front end of the second wire.

2. The bipolar electrode type guide wire according to claim 1, further comprising a spherical first electrode formed of an electrically conductive material and coupled to the front end of the first wire.

3. The bipolar electrode type guide wire according to claim 2, wherein the first spherical electrode has the same diameter as an outer diameter of the coil part of the second wire.

4. The bipolar electrode type guide wire according to claim 1, further comprising a ring-shaped second electrode formed of an electrically conductive material and coupled to the front end of the second wire.

5. The bipolar electrode type guide wire according to claim 1, wherein a spacing distance between the front ends of the first and second wires is 1 mm to 50 mm.

6. The bipolar electrode type guide wire according to claim 1, wherein one end of the first wire is bent in a direction perpendicular to a longitudinal direction of the catheter.

7. The bipolar electrode type guide wire according to claim 1, wherein one end of the first wire is bent to form a “U” shape.

8. The bipolar electrode type guide wire according to claim 1, wherein an outer periphery of the first wire is coated with a Teflon coating material to electrically insulate the first wire from the second wire.

9. The bipolar electrode type guide wire according to claim 1, wherein an outer periphery of the main wire part of the second wire is coated with a Teflon coating material.

10. The bipolar electrode type guide wire according to claim 8, further comprising a spherical first electrode formed of an electrically conductive material and coupled to the front end of the first wire, and a ring-shaped second electrode formed of an electrically conductive material and coupled to the front end of the second wire,
wherein the thickness of the coating material coated on the first wire, which is disposed between the first electrode and the second electrode, is equal to the outer diameter of the coil part of the second wire.

11. The bipolar electrode type guide wire according to claim 1, wherein the first wire and the main wire part of the second wire are electrically insulated by an insulating material interposed between the first wire and the second wire, and are coupled to each other to prevent relative movement therebetween.

12. The bipolar electrode type guide wire according to claim 1, wherein the first wire and the main wire part of the second wire are spaced apart from each other and surrounded by a covering material formed of an insulating material to be physically connected with each other, and
the covering material is interposed between the first wire and the main wire part of the second wire to electrically insulate the first wire from the main wire part of the second wire.

13. A catheter system comprising:
a hollow catheter inserted into a fistula of a living body, the catheter having two open ends and a coaxially-formed lumen; and
a bipolar electrode type guide wire including a first wire formed of an electrically conductive material, having an elongated shape, and inserted into the lumen of the catheter to project from the lumen at both ends thereof, and a second wire formed of an electrically conductive material, and including a main wire part spaced apart from the first wire having an elongated shape, and a coil part extending from the main wire part in a spiral shape, the first wire being inserted into the coil part,
wherein the first wire is electrically insulated from the second wire, front ends of the first and second wires are not insulated, and the non-insulated front end of the first wire is spaced a predetermined distance apart from the non-insulated front end of the second wire.

14. The catheter system according to claim 13, further comprising a radio-frequency generator for generating radio-frequency current,
wherein the first wire and the second wire of the bipolar electrode type guide wire are electrically connected to the radio-frequency generator and have different polarities.

15. A bipolar electrode type guide wire comprising:
a wire body formed of a metal and elongated in one direction to be inserted into a fistula of a living body;
an insulating material coated on an outer periphery of the wire body to insulate the wire body;
a first electrode attached to one end of the wire body;
a second electrode attached to the end of the wire body and spaced a predetermined distance apart from the first electrode;
a first lead elongated along the wire body to be attached to the first electrode to electrically connect a radio-frequency generator for generating radio-frequency current to the first electrode; and
a second lead elongated along the wire body to be attached to the second electrode to electrically connect the radio-frequency generator to the second electrode, the second lead being electrically insulated from the first lead.

16. The bipolar electrode type guide wire according to claim 15, wherein the first electrode and the second electrode have a ring shape to be fit and attached onto the outer periphery of the wire body.

17. The bipolar electrode type guide wire according to claim 15, wherein a front end of the wire body is bent.

18. The bipolar electrode type guide wire according to claim 17, wherein the front end of the wire body is bent to form a “U” shape.

19. The bipolar electrode type guide wire according to claim 15, further comprising an insulator disposed between the first electrode and the second electrode.

20. The bipolar electrode type guide wire according to claim 15, wherein a spacing distance between the first electrode and the second electrode is 1 mm to 50 mm.

21. The bipolar electrode type guide wire according to claim 15, wherein the first electrode and the second electrode have a ring shape, are fit onto the outer periphery of the wire body, and have the same outer diameter, and an insulator having the same outer diameter as the first electrode and the second electrode is inserted between the first electrode and the second electrode to prevent generation of a step between the first electrode and the second electrode, which are spaced apart from each other.

22. A catheter system comprising:
a hollow catheter inserted into a fistula of a living body, the catheter having two open ends and a coaxially-formed lumen; and
a bipolar electrode type guide wire including a wire body formed of a metal and elongated in one direction to be inserted into the catheter, an insulating material coated on an outer periphery of the wire body to insulate the wire body, a first electrode attached to one end of the wire body, a second electrode attached to the end of the wire body and spaced a predetermined distance apart from the first electrode, a first lead elongated along the wire body to be attached to the first electrode to electrically connect a radio-frequency generator for generating radio-frequency current to the first electrode, and a second lead elongated along the wire body to be attached to the second electrode to electrically connect the radio-frequency generator to the second electrode, the second lead being electrically insulated from the first lead.

23. A catheter system comprising:
a hollow catheter inserted into a fistula of a living body, formed of an insulating material, the catheter having two open ends and a coaxially-formed lumen;
a shape holding body for an electrode elongated in a longitudinal direction of the catheter and inserted between an inner periphery and an outer periphery of the catheter to maintain the shape of the lumen to support the catheter, and formed of an electrically conductive material, both ends of which project from the catheter to be exposed to the exterior; and
a guide wire formed of an electrically conductive material, and coaxially inserted into the lumen such that one end thereof projects from the lumen to be spaced a predetermined distance apart from one end of the shape holding body for an electrode.

24. The catheter system according to claim 23, wherein the shape holding body for an electrode is formed in a spiral shape to be wound between the inner periphery and the outer periphery of the catheter.

25. The catheter system according to claim 23, wherein the shape holding body for an electrode has a net shape.

26. The catheter system according to claim 23, wherein the shape holding body for an electrode comprises a plurality of annular support bodies disposed in a longitudinal direction of the catheter at predetermined intervals, and a plurality of linear support bodies disposed along a circumference of the annular support bodies, each of which is elongated in the longitudinal direction of the catheter to be connected to the annular support body, such that both ends of the linear support body project from the catheter to be exposed to the exterior.

27. The catheter system according to claim 23, wherein the shape holding body for an electrode is elongated in the longitudinal direction of the catheter, and at least one shape holding body for an electrode is disposed between the inner periphery and the outer periphery of the catheter.

28. The catheter system according to claim 23, further comprising a ring-shaped electrode member formed of an electrically conductive material and fit onto the outer periphery of one end of the catheter to be coupled thereto,
wherein the shape holding body for an electrode is electrically connected to the ring-shaped electrode member.

29. The catheter system according to claim 28, wherein the ring-shaped electrode member is spaced a predetermined distance apart from a tip of one end of the catheter toward the other end thereof.

30. The catheter system according to claim 23, wherein the length of the one end of the guide wire projecting from the lumen is 0.5 to 2 cm and a center part between the one end and the other end of the guide wire is coated with an insulating material formed of a polymer material.

31. The catheter system according to claim 23, further comprising a radio-frequency generator for generating radio-frequency current,
wherein the other end of the shape holding body for an electrode and the other end of the guide wire are electrically connected to the radio-frequency generator and have different polarities.

32. The catheter system according to claim 23, wherein the lumen includes a first lumen configured to pass through the catheter form the one end to the other end thereof, and a second lumen isolated form the first lumen by a diaphragm, one end of which passes through between the inner periphery and the outer periphery of the catheter to be in communication with the exterior of the catheter, further comprising a hollow fluid injection tube including a balloon closely attached to the outer periphery of the catheter to surround the one end of the second lumen to expand when fluid is injected through the second lumen, and a third lumen formed therein and connected to the second lumen to guide the fluid injected into the balloon to the second lumen of the catheter.

33. The catheter system comprising:
a hollow catheter inserted into a fistula of a living body, formed of an insulating material, and having two open ends and a coaxially-formed lumen; and
a shape holding body for an electrode elongated in a longitudinal direction of the catheter and inserted between an inner periphery and an outer periphery of the catheter to maintain the shape of the lumen to support the catheter body, and formed of an electrically conductive material, both ends of which project from the catheter to be exposed to the exterior.

34. The catheter system according to claim 33, further comprising a ring-shaped electrode member formed of an electrically conductive material and fit onto and coupled with an outer periphery of the one end of the catheter,
wherein the shape forming body for an electrode is electrically connected to the ring-shaped electrode member.
